(19) United States
(12) Patent Application Publication
(10) Pub. No.: US 2008/0227882 A1
(43) Pub. Date: Sep. 18, 2008

(54) Multifunctional Monomers Containing Bound Poragens and Polyarylene Compositions Therefrom

(76) Inventors: Jerry L. Hahnfeld, Midland, MI (US); John W. Lyons, Midland, MI (US); Q. Jason Niu, Excelsior, MN (US)

Correspondence Address: The Dow Chemical Company, Intellectual Property Section, P.O. Box 1967, Midland, MI, 48641-1967, US

(21) Appl. No.: 10/571,795

(22) PCT Filed: Sep. 15, 2004
(86) PCT No.: PCT/US2004/030228

§ 371 Date: Mar. 15, 2006

Related U.S. Application Data
(60) Provisional application No. 60/504,420, filed on Sep. 19, 2003.

(51) Int. Cl. C08G 16/00; C07C 49/297

(52) U.S. Cl. 521/189; 568/330; 528/220

(57) ABSTRACT

A compound (monomer) comprising i) one or more dienophile groups (A-functional groups), ii) one or more ring structures comprising two conjugated carbon-to-carbon double bonds and a leaving group L (B-functional groups), and iii) one or more chemically bound poragens, characterized in that the A-functional group of one monomer is capable of reaction under cycloaddition reaction conditions with the B-functional group of a second monomer to thereby form a cross-linked, polyphenylene polymer.

### FIELD OF THE INVENTION

This invention relates to compositions comprising bound poragen moieties and having at least two different reactive functional groups and to aromatic polymers made from these monomers. More particularly, the invention relates to compositions comprising in a single monomer polyphenylene matrix forming functionality and a poragen. The resulting polymers are useful in making low dielectric constant insulating layers in microelectronic devices.

### BACKGROUND OF THE INVENTION

Polyarylene resins, such as those disclosed in U.S. Pat. No. 5,965,679 (Godschalx et al.) are low dielectric constant materials suitable for use as insulating films in semiconductor devices, especially integrated circuits. Such polyarylene compounds are prepared by reacting polyfunctional compounds having two or more cyclopentadienone groups with polyfunctional compounds having two or more aromatic acetylene groups, at least some of the polyfunctional compounds having three or more reactive groups. Certain single component reactive monomers which contained one cyclopentadienone group together with two aromatic acetylene groups, specifically 3,4-bis(3-(phenylethynyl)phenyl)-2,5-dicyclopentadienone and 3,4-bis(4-(phenylethynyl)phenyl)-2,5-dicyclopentadienone, and polymers made from such monomers were also disclosed in the foregoing reference. Typically, these materials are b-staged in a solution and then coated onto a substrate followed by curing (vitrification) at elevated temperatures as high as 400-450° C. to complete the cure.

In U.S. Pat. No. 6,359,091, it was taught that it may be desirable to adjust the modulus of polymers as taught in Godschalx et al., by adjusting the ratio of the reactants or by adding other reactive species to the monomers or to the partially polymerized product of Godschalx et al. U.S. Pat. No. 6,172,128 teaches aromatic polymers containing cyclopentadienone groups that may react with aromatic polymers containing phenylacetylene groups to provide branched or cross-linked polymers. U.S. Pat. No. 6,156,812 discloses polymers which contain both cyclopentadienone- and phenyl acetylene-backbone groups.

In WO 00/31183, cross-linkable compositions comprising a cross-linkable hydrocarbon-containing matrix precursor and a separate pore forming substance (poragen) which are curable to form low dielectric constant insulating layers for semiconductor devices were disclosed. By partially, curing the precursor to form a matrix containing occlusions of the poragen and then removing the pore generating material to form voids or pores in the matrix material, lower dielectric constant insulating films may be prepared. It has now been discovered that the use of mixtures of a curable matrix resin and a separately added pore forming material, especially an ultra-small sized poragen, to form a b-staged polyphenylene resin formulation can suffer from poragen agglomeration, resulting in large diameter pore formation and an inhomogeneous distribution of pores, leading to variation in the electronic properties of the resulting film.

Although the foregoing advances have led to improvements in dielectric constant of the resulting film, additional improvements in film properties are desired by the industry. In particular, curable compositions capable of providing homogeneous, porous matrices by means of a single component are still desired. In addition, films and other cured compositions having improved physical properties, especially uniformly distributed, small pores, are sought.

### SUMMARY OF THE INVENTION

According to a first embodiment of the present invention there is provided a compound (monomer) comprising i) one or more dienophile groups (A-functional groups), ii) one or more ring structures comprising two conjugated carbon-to-carbon double bonds and a leaving group L (B-functional groups), and iii) one or more chemically bound poragens, characterized in that the A-functional group of one monomer is capable of reaction under cycloaddition reaction conditions with the B-functional group of a second monomer to thereby form a cross-linked, polyphenylene polymer.

According to a second embodiment of this invention, there is provided a curable oligomer or polymer made by the partial reaction of the A and B groups of the foregoing monomer, a mixture thereof, or a composition comprising the same under cycloaddition reaction conditions. In this embodiment of the invention the curable oligomer or polymer comprises some remainder of the two reactive A and B functional groups as pendant groups, terminal groups, or as groups within the backbone of the oligomer or polymer.

According to a third embodiment of the invention, there is provided a crosslinked polymer made by curing and crosslinking the foregoing curable monomers, oligomers or polymers of the first or second embodiments, or compositions comprising the same. Desirably, the resulting cross-linked polymer contains bound poragens that are homogeneously distributed throughout the polymer.

According to a fourth embodiment of the invention there is provided a process for making a porous, solid article comprising a vitrified polyarylene polymer which process comprises providing the foregoing curable monomers or oligomers of the first through third embodiments, or polymers or compositions comprising the same; partially polymerizing the monomer under cycloaddition reaction conditions optionally in the presence of a solvent and/or one or more separately added poragens, thereby forming a curable oligomer or polymer containing composition; and curing and crosslinking the composition to form a solid polyarylene polymer containing bound porogens and optionally separately added poragens. In a further step, the optional solvent, bound poragens, and/or separately added poragens may be removed.

In a fifth embodiment, this invention is an article made by the above method, desirably a porous article formed by removal of bound porogens and/or separately added poragens. Desirably, said article contains a homogeneous distribution of pores.

According to a sixth embodiment of the invention, the foregoing article is a film or a construct such as a semiconductor device incorporating the film as an insulator between circuit lines or layers of circuit lines therein.

The monomers are highly soluble in typical solvents used in fabrication of semiconductor devices, and may be employed in formulations that are coated onto substrates and vitrified to form films and other articles. The compositions are desirable in order to obtain films having uniformly distributed small pores having a reduced potential for pore collapse or coalescence during the chip manufacturing process, and accordingly uniform electrical properties, and low dielectric constants.

### DETAILED DESCRIPTION OF THE INVENTION

For purposes of United States patent practice, the contents of any patent, patent application or publication referenced herein is hereby incorporated by reference in its entirety herein, especially with respect to its disclosure of monomer, oligomer or polymer structures, synthetic techniques and general knowledge in the art. If appearing herein, the term “comprising” and derivatives thereof is not intended to exclude the presence of any additional component, step or procedure, whether or not the same is disclosed herein. In order to avoid any doubt, all compositions claimed herein through use of the term “comprising” may include any additional additive, adjuvant, or compound, unless stated to the contrary. In contrast, the term, “consisting essentially of” if appearing herein, excludes from the scope of any succeeding recitation any other component, step or procedure, excepting those that are not essential to operability. The term “consisting of”, if used, excludes any component, step or procedure not specifically delineated or listed. The term “or”, unless apparent from the context or stated otherwise, refers to the listed members individually as well as in any combination.

As used herein the term “aromatic” refers to a polyatomic, cyclic, ring system containing (4δ+2) π-electrons, wherein δ is an integer greater than or equal to 1. The term “fused” as used herein with respect to a ring system containing two or more polyatomic, cyclic rings means that with respect to at least two rings thereof, at least one pair of adjacent atoms is included in both rings.

“A-functionality” refers to a single dienophile group.

“B-functionality” refers to the ring structure comprising two conjugated carbon-to-carbon double bonds and a leaving group L.

“b-staged” refers to the oligomeric mixture or low molecular weight polymeric mixture resulting from partial polymerization of a monomer or monomer mixture. Unreacted monomer may be included in the mixture.

“Cross-linkable” refers to a matrix precursor that is capable of being irreversibly cured, to a material that cannot be reshaped or reformed. Cross-linking may be assisted by thermal, UV, microwave, x-ray, or e-beam irradiation.

“Dienophile” refers to a group that is able to react with the conjugated, double bonded carbon groups according to the present invention, preferably in a cycloaddition reaction involving elimination of the L group and aromatic ring formation.

“Inert substituent” means a substituent group which does not interfere with any subsequent desirable polymerization reaction of the monomer or b-staged oligomer and does not include further polymerizable moieties as disclosed herein.

“Matrix precursor” means a monomer, prepolymer, polymer, or mixture thereof which upon curing or further curing forms a cross-linked polymeric material.

“Monomer” refers to a polymerizable compound or mixture thereof.

“Matrix” refers to a continuous phase surrounding dispersed regions of a distinct composition or void.

“Poragen” refers to polymeric or oligomeric components that may be combined with the monomers, oligomers, or polymers of the invention, and which may be removed from the initially formed oligomer or, more preferably, from the vitrified (that is the fully cured or cross-linked) polymer matrix, resulting in the formation of voids or pores in the polymer. Poragens may be removed from the matrix polymer by any suitable technique, including dissolving with solvents or, more preferably, by thermal decomposition. A “bound poragen” refers to a poragen that is chemically bound or grafted to the monomer, oligomer, or vitrified polymer matrix.

### The Monomers and Their Syntheses

The monomers of the present invention preferably comprise one or more dienophilic functional groups, preferably an arylacetylenic group; one or more hydrocarbon—or heteroatom substituted hydrocarbon—rings having two conjugated carbon to carbon double bonds and the leaving group, L; one or more bound poragen side chains; and, optionally, inert substituents. Desirably, the poragen side chains are bound to a moiety comprising a B-functionality through an A-functional group (

Preferred B-functional groups comprise cyclic, five-membered, conjugated diene rings where L is —O—, —S—, —(CO)—, or —(SO₂)—, or a six membered, conjugated diene ring where L is —N═N—, or —O(CO)—. Optionally, two of the carbon atoms of the ring structure and their substituent groups taken together may also form an aromatic ring, that is, the 5 or 6 membered ring structures may be part of a fused, multiple aromatic ring system.

Most preferably, L is —(CO)— such that the ring is a cyclopentadienone group or benzcyclopentadienone group. Examples of such most preferred cyclopentadienone rings are those containing aryl groups at the 2, 3, 4, or 5 positions thereof, more preferably at the 2, 3, 4 and 5 positions thereof.

Preferred dienophile groups (A-functionality) are unsaturated hydrocarbon groups, most preferably ethynyl or phenylethynyl groups.

The monomers of the present invention may be depicted generically by the formula: AxByP*z, wherein A, B and P* stand for A-functionality, B-functionality and poragen side chain respectively, and x, y and z are integers greater than or equal to one. More preferably, x is greater than or equal to 2, and y and z are greater than or equal to 2.

Examples of suitable monomers according to the invention are compounds corresponding to the formula,

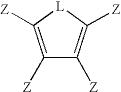

wherein L is —O—, —S—, —N═N—, —(CO)—, —(SO₂)—, or —O(CO)—;

Z is independently in each occurrence hydrogen, halogen, an unsubstituted or inertly substituted hydrocarbyl group, especially an aryl group, more especially a phenyl group, Z″, or two adjacent Z groups together with the carbons to which they are attached form a fused aromatic ring,

Z″ is a divalent derivative of an unsubstituted or inertly substituted hydrocarbyl group joining two or more of the foregoing structures, or joining an A-functionality, a bound poragen and/or a combination of the foregoing,

and in at least one occurrence, Z is -Z″-C≡CP*;

or

in at least one occurrence, Z is -Z″-C≡CR and in at least one other occurrence Z is a bound poragen; wherein,

P* is independently each occurrence a bound poragen; and

R is independently each occurrence selected from the group consisting of hydrogen, C₁₋₄ alkyl, C₆₋₆₀ aryl, and C₇₋₆₀ inertly substituted aryl groups.

Preferred monomers according to the present invention are 3-substituted cyclopentadienone compounds or 3,4-disubstituted cyclopentadienone compounds, represented by the formula:

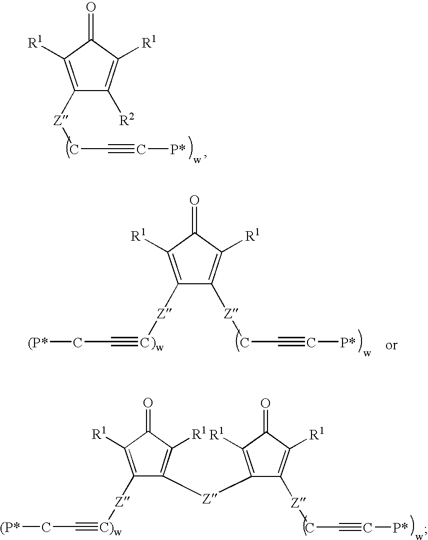

wherein R¹ is P*, C₆₋₂₀ aryl, inertly substituted aryl, or R²OC(O)—, more preferably, phenyl, biphenyl, p-phenoxyphenyl or naphthyl,

R² is P*, C₆₋₂₀ aryl, inertly substituted aryl, more preferably, phenyl, biphenyl, p-phenoxyphenyl, or naphthyl;

w independently each occurrence is an integer from 1 to 3, more preferably 1,

Z″ is a divalent aromatic group, more preferably phenylene, biphenylene, phenyleneoxyphenylene, and

P* is a bound poragen, preferably a monovalent derivative of a linear or branched oligomer or polymer of a vinylaromatic monomer, alkylene oxide, arylene oxide, alkylacrylate or alkylmethacrylate, or a cross-linked derivative thereof.

Highly preferred examples of the foregoing monomers are represented by the following structures:

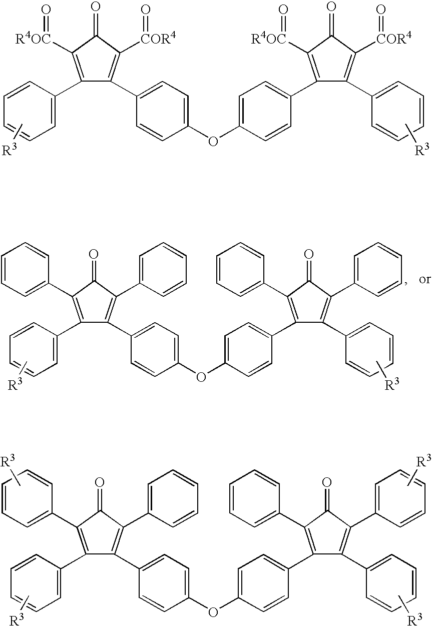

wherein R³ each occurrence is —C≡C—P*, and

R⁴ independently each ocurrence is H, phenyl or P*.

### Synthesis of AxByP*z Monomers

The monomers according to the present invention may be made by the condensation of diaryl-substituted acetone compounds with aromatic polyketones using conventional methods. Exemplary methods are disclosed in Macromolecules, 28, 124-130 (1995); J. Org. Chem, 30, 3354 (1965); J. Org. Chem., 28, 2725 (1963); Macromolecules, 34, 187 (2001); Macromolecules 12, 369 (1979); J. Am. Chem. Soc. 119, 7291 (1997); and U.S. Pat. No. 4,400,540.

More preferably, the monomers may be made by the condensation of the following synthons, or molecular components, according to one of the following schemes:

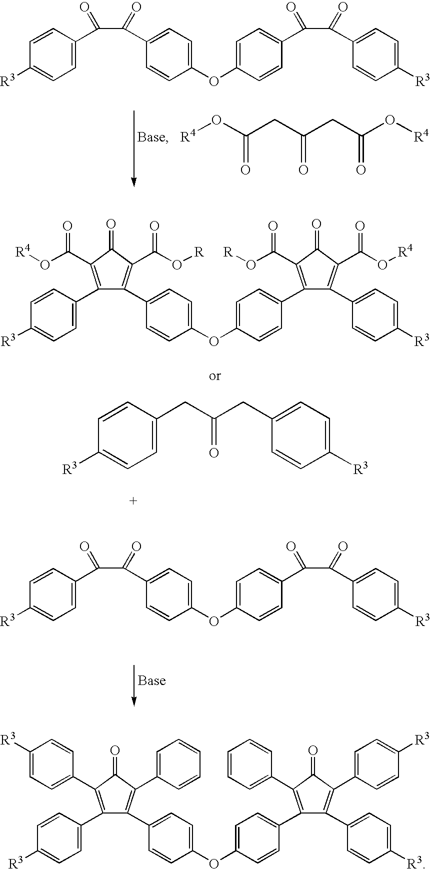

### B-Staging of AxByP*z Monomer

Preparation of oligomers and partially cross-linked polymers (b-staging) can be represented in one embodiment employing an A₂B₂P*₂ monomer by the following illustration, where XL stands for a cross-linking polymer chain. A variety of similarly cross-linked polymers may be prepared by this technique.

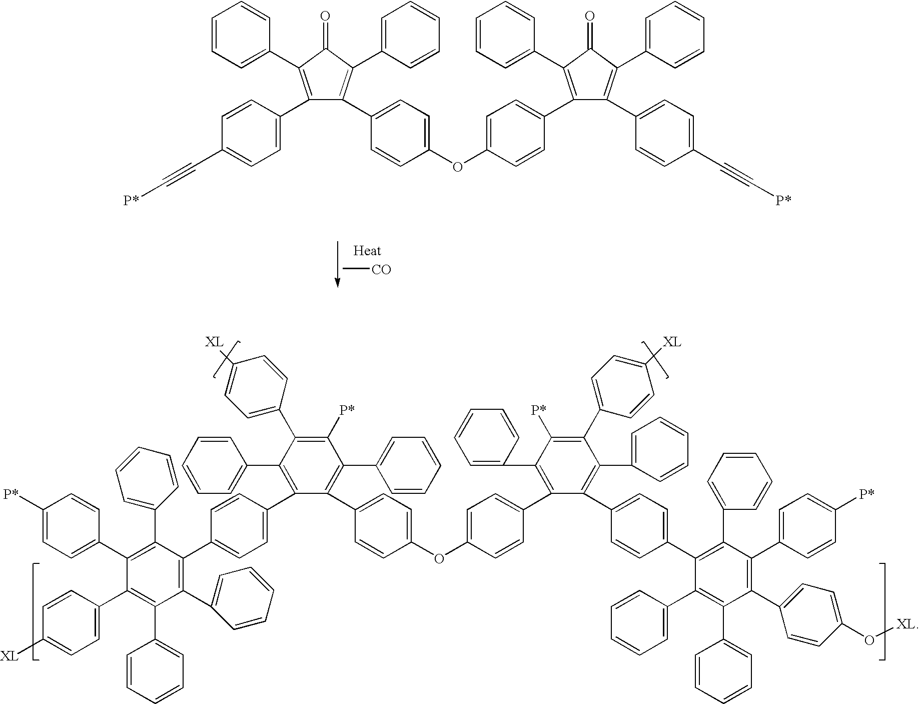

While not desiring to be bond by their belief, it is believed that polyphenylene oligomers and polymers are formed through a Diels-Alder reaction of the cyclopentadienone with the acetylene group when the mixture of monomer and an optional solvent is heated. The product may still contain quantities of cyclopentadienone and acetylene end groups. Upon further heating of the mixture or an article coated therewith, additional crosslinking can occur through the Diels-Alder reaction of the remaining cyclopentadienone or B groups with the remaining acetylene or A groups. Ideally, cyclopentadienone and acetylene groups are consumed at the same rate under Diels-Alder reaction conditions, preferably at temperatures from 280 to 350° C., more preferably from 285 to 320° C.

The cross-linking reaction is preferably halted prior to the reaction of significant quantities of A and B functionality to avoid gel formation. The oligomer may then be applied to a suitable surface prior to further advancement or curing of the composition. While in an oligomerized or b-stage, the composition is readily applied to substrates by standard application techniques, and forms a level surface coating which covers (planerizes) components, objects or patterns on the surface of the substrate. Preferably, at least ten percent of the monomer remains unreacted when b-staged. Most preferably, at least twenty percent of the monomer remains unreacted. One may determine the percentage of unreacted monomer by visible spectra analysis or SEC analysis.

Suitable solvents for preparing coating compositions of b-staged compositions include mesitylene, methyl benzoate, ethyl benzoate, dibenzylether, diglyme, triglyme, diethylene glycol ether, diethylene glycol methyl ether, dipropylene glyco methyl ether, dipropylene glycol dimethyl ether, propylene glycol methyl ether, dipropylene glycol monomethyl ether acetate, propylene carbonate, diphenyl ether, butyrolactone. The preferred solvents are mesitylene, gamma-butyrolactone, diphenyl ether and mixture thereof.

Alternatively, the monomers can be polymerized in one or more solvents at elevated temperature and the resulting solution of oligomers can be cooled and formulated with one or more additional solvents to aid in processing. In another approach, the monomer can be polymerized in one or more solvents at elevated temperature to form oligomers which can be isolated by precipitation into a non solvent. These isolated oligomers can then be redissolved in a suitable solvent for processing.

The monomers of the present invention or b-staged oligomers thereof are suitably employed in a curable composition alone or as a mixture with other monomers containing two or more functional groups (or b-staged oligomers thereof) able to polymerize by means of a Diels-Alder or similar cycloaddition reaction. Examples of such other monomers include compounds having two or more cyclopentadienone functional groups and/or acetylene functional groups or mixtures thereof, such as those previously disclosed in U.S. Pat. Nos. 5,965,679 and 6,359,091. In the b-stage curing reaction, a dienophilic group reacts with the cyclic diene functionality, causing elimination of L and aromatic ring formation. Subsequent curing or vitrification may involve a similar cycloaddition or an addition reaction involving only the dienophilic functional groups.

Additional polymerizable monomers containing A and/or B functionality may be included in a curable composition according to the present invention. Examples include compounds of the formula:

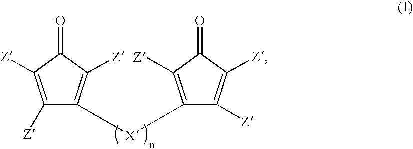

wherein

Z′ is independently in each occurrence hydrogen, an unsubstituted or inertly substituted aromatic group, an unsubstituted or inertly substituted alkyl group, or —W—(C≡C-Q)_{q};

X′ is an unsubstituted or inertly substituted aromatic group, —W—C≡C—W—, or

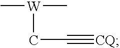

W is an unsubstituted or inertly substituted aromatic group, and

Q is hydrogen, an unsubstituted or inertly substituted C₆₋₂₀ aryl group, or an unsubstituted or inertly substituted C₁₋₂₀ alkyl group, provided that at least two of the X′ and/or Z′ groups comprise an acetylenic group,

q is an integer from 1 to 3; and

n is an integer of from 1 to 10.

Examples of the foregoing polyfunctional monomers that may be used in conjunction with the monomers of the present invention include compounds of formulas II-XXV:

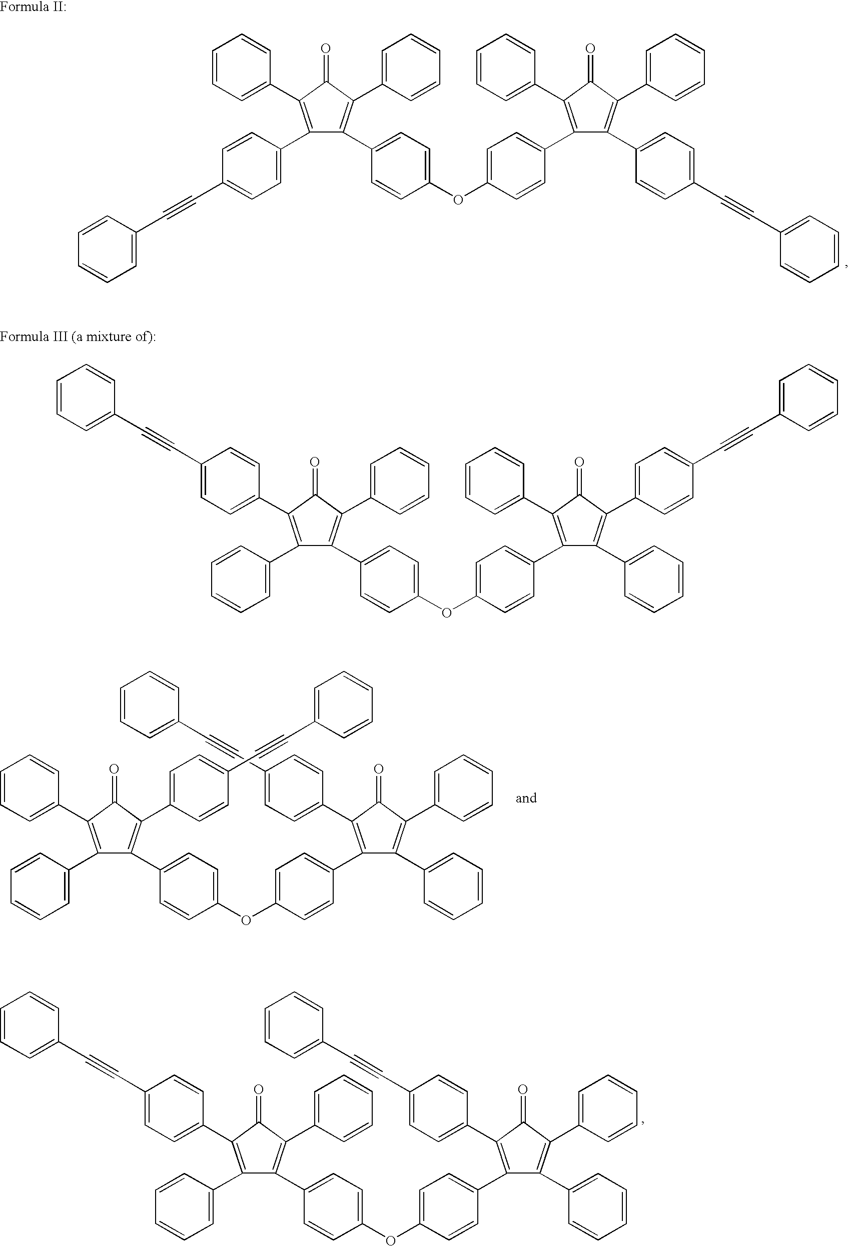

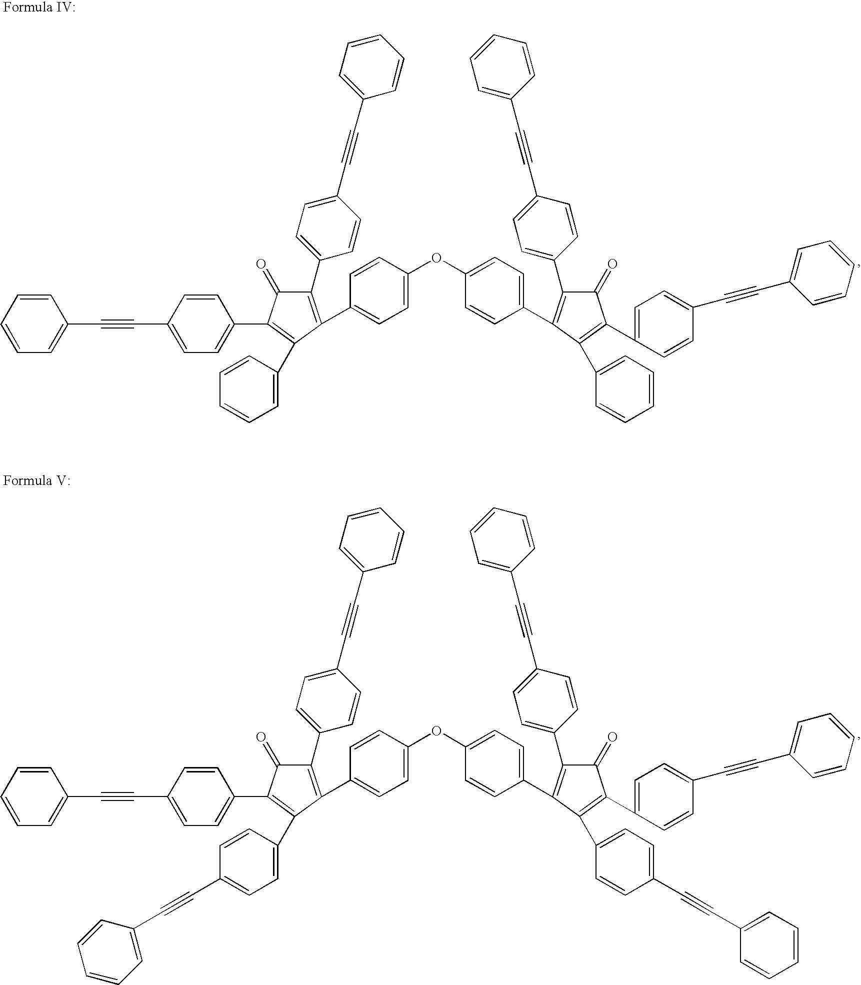

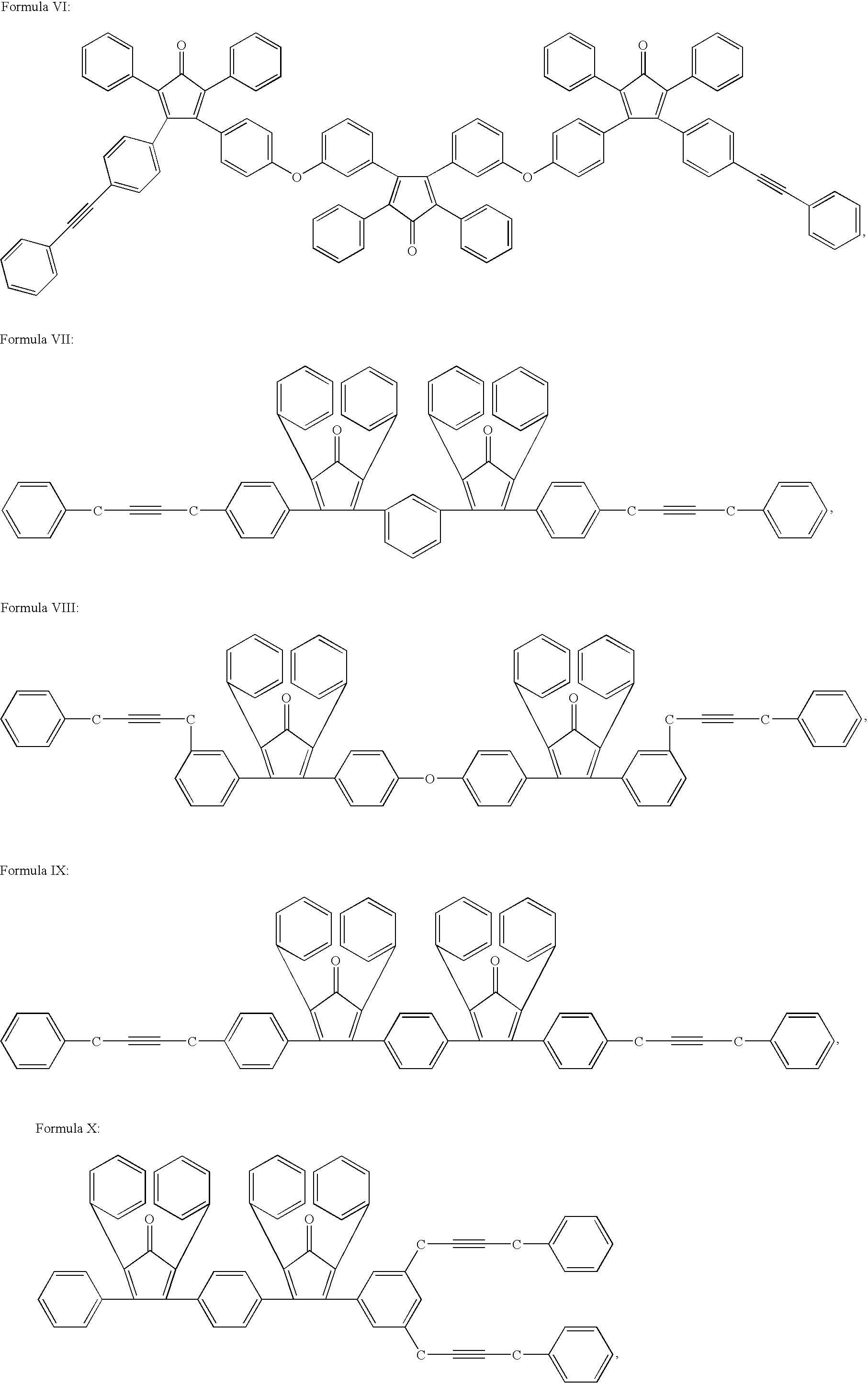

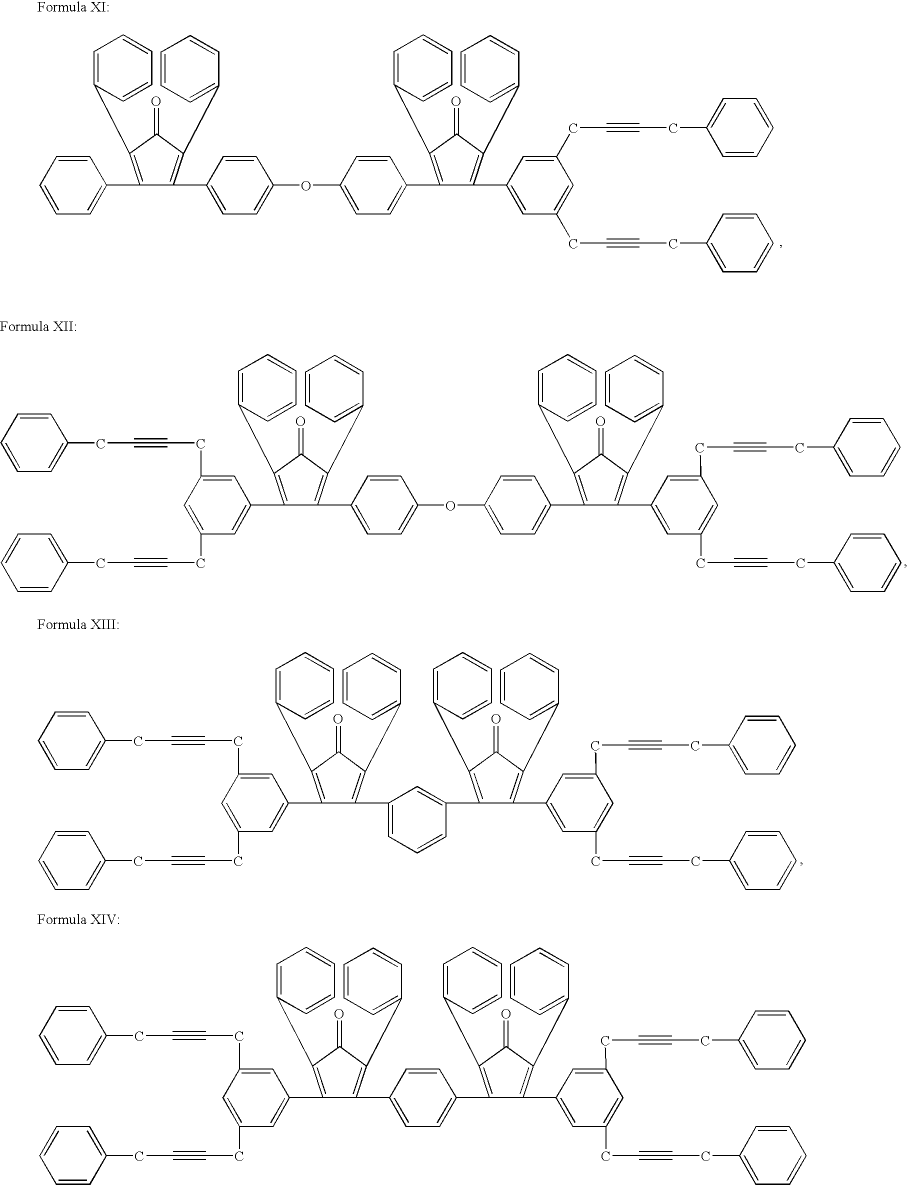

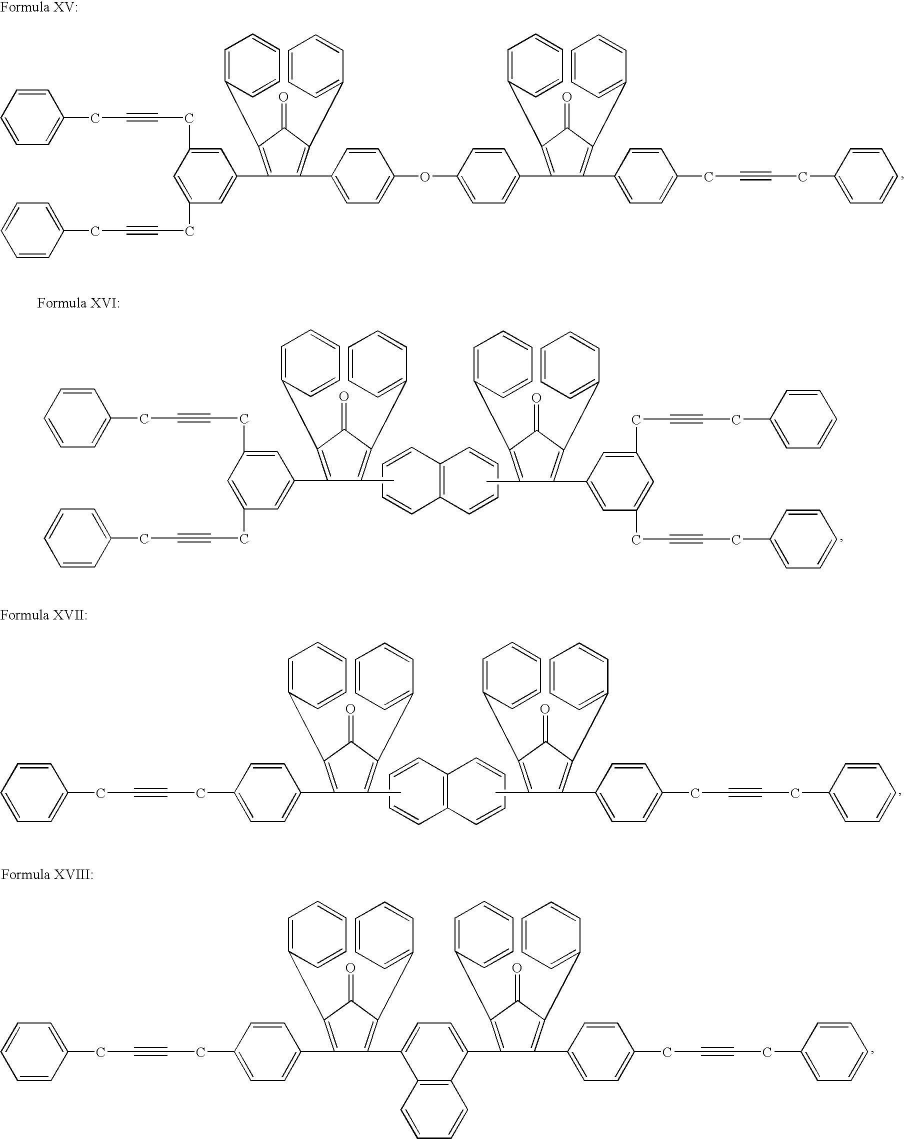

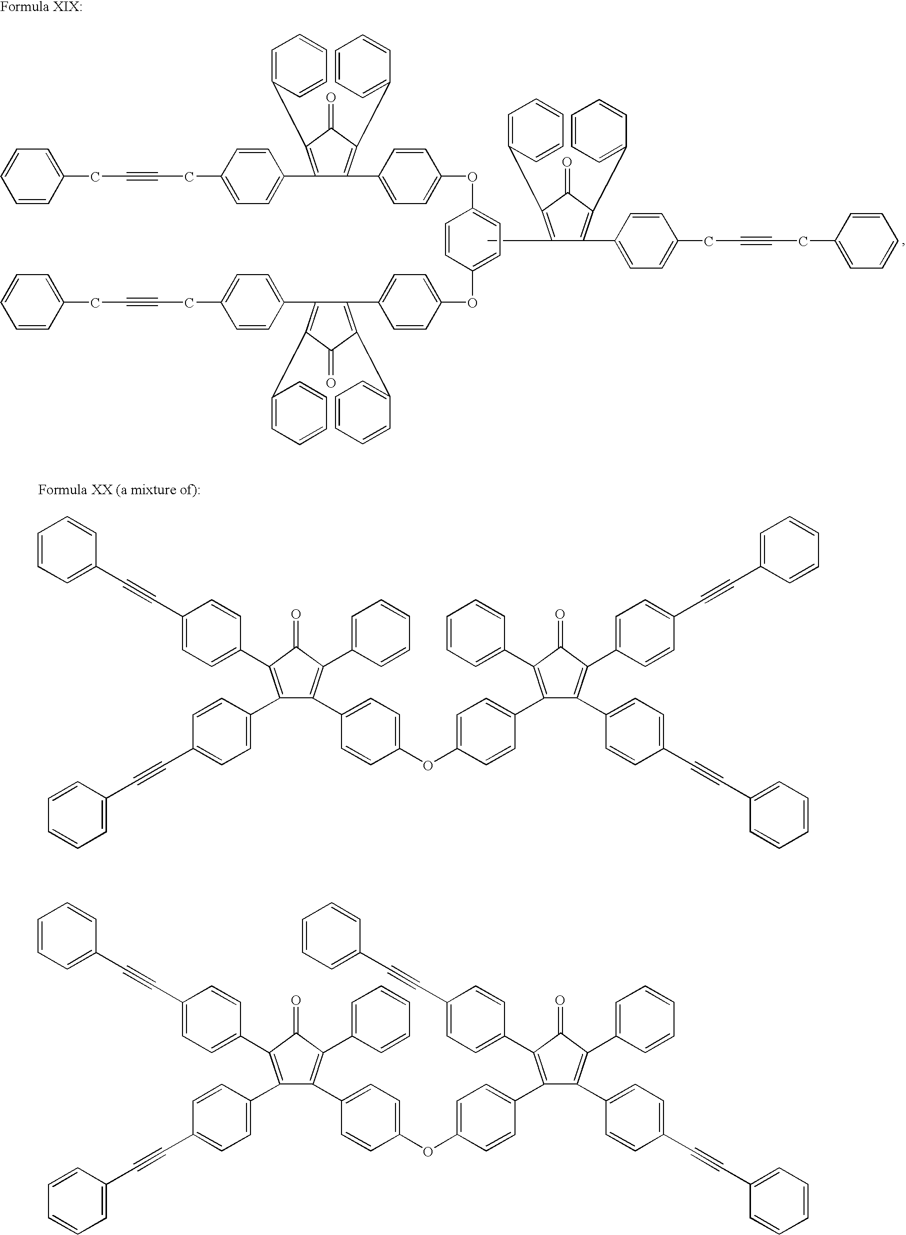

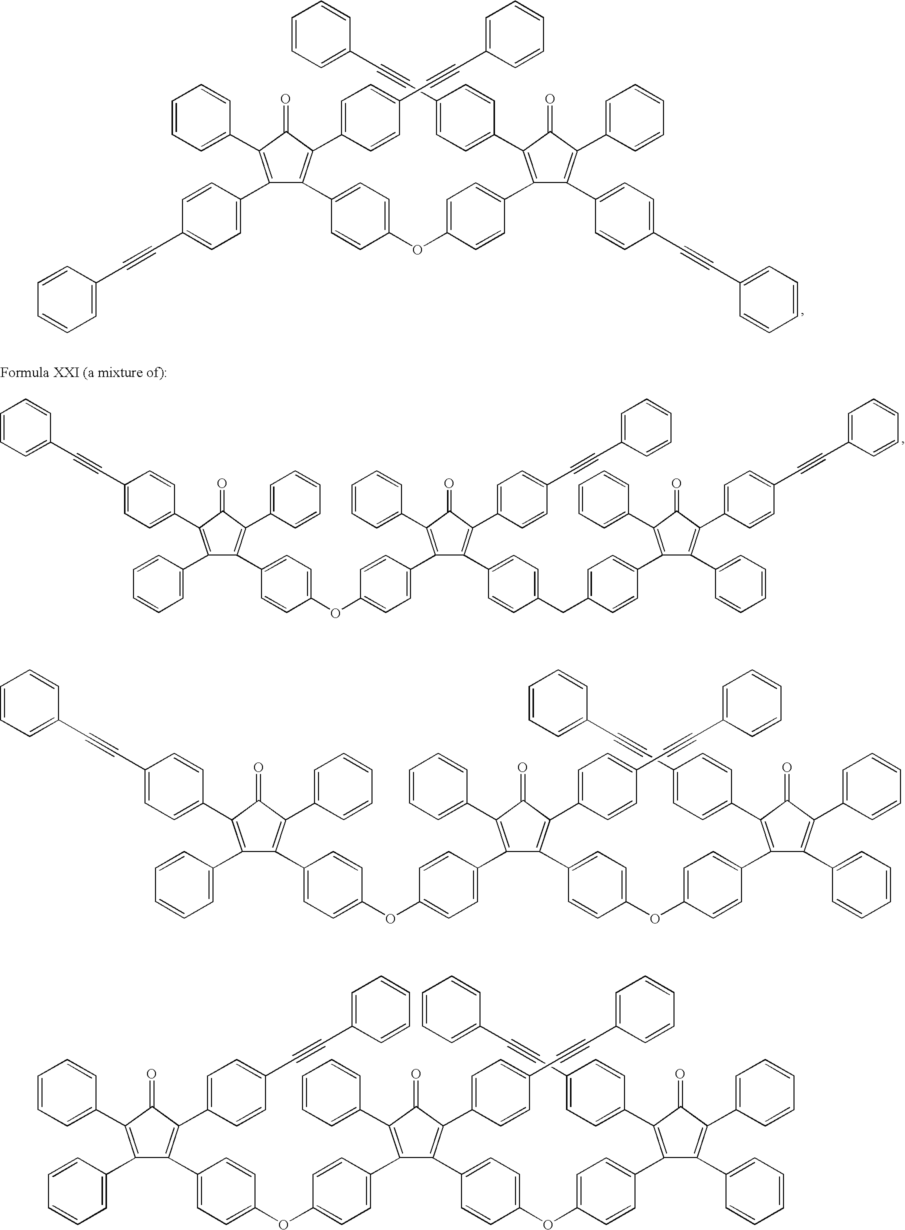

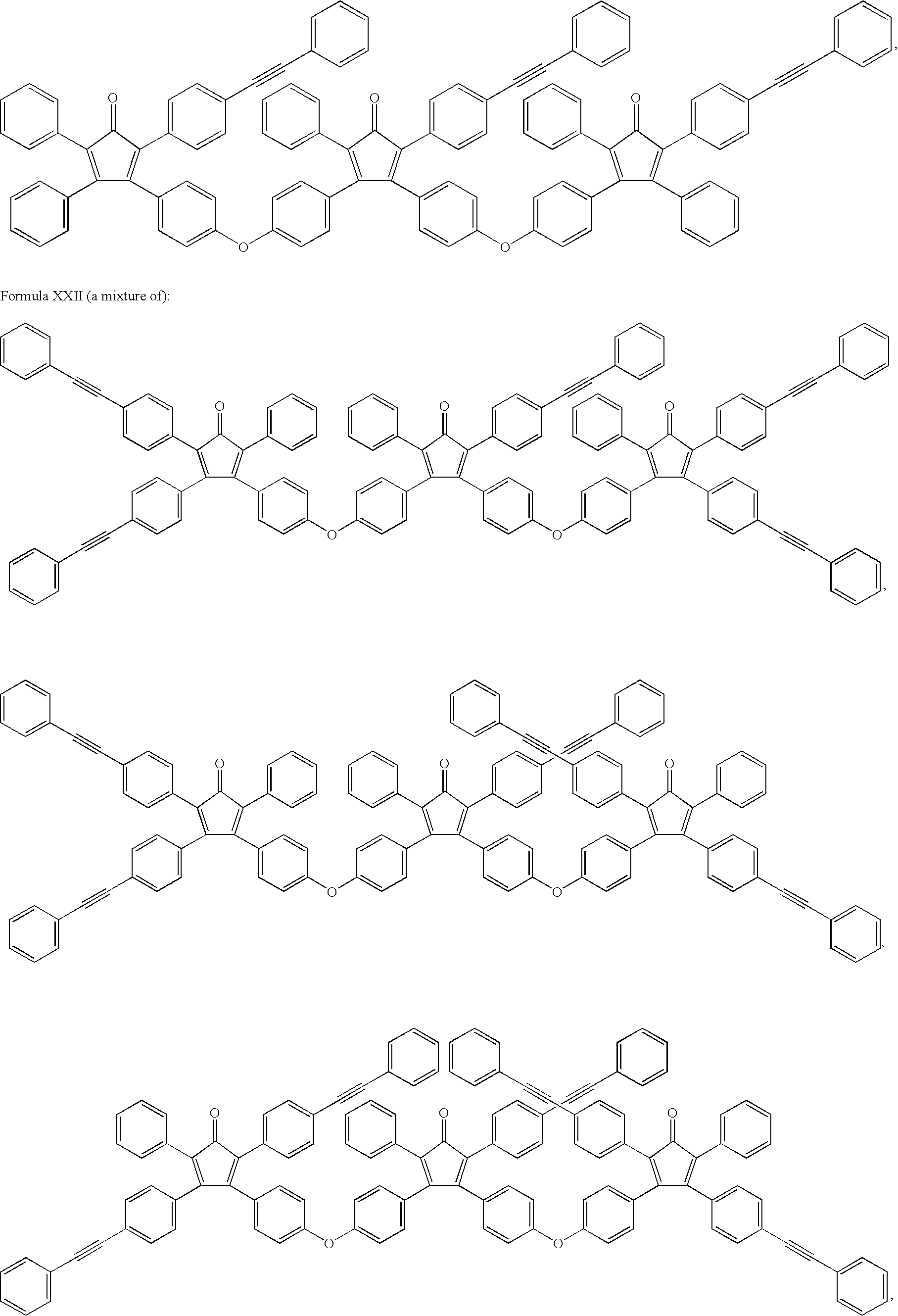

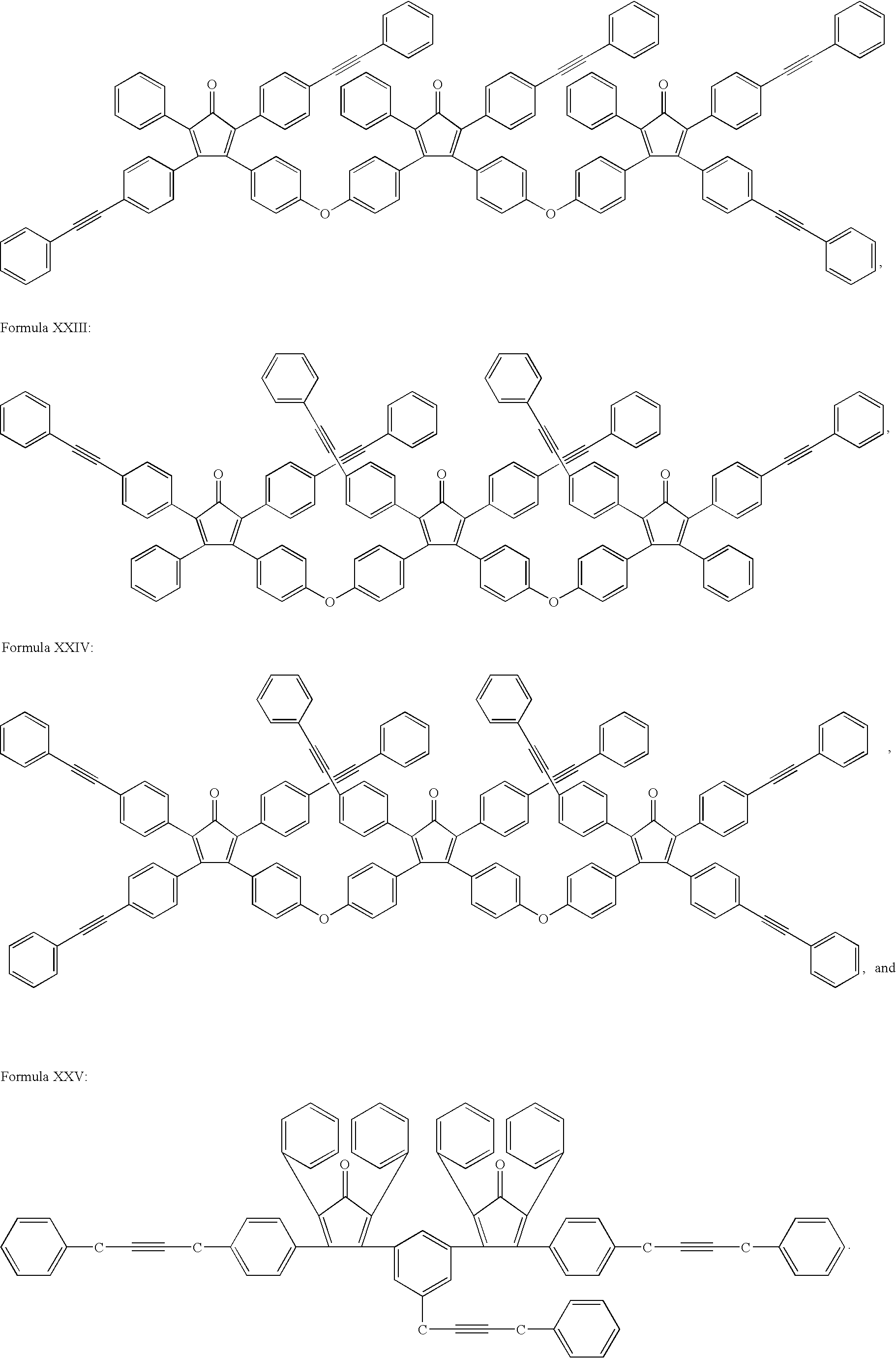

The foregoing monomers I-XXV where the ring structure is a cyclopentadienone may be made, for example, by condensation of substituted or unsubstituted benzils with substituted or unsubstituted benzyl ketones (or analogous reactions) using conventional methods such as those previously disclosed with respect to AxByC′z monomers. Monomers having other structures may be prepared as follows: Pyrones can be prepared using conventional methods such as those shown in the following references and references cited therein: Braham et. al., Macromolecules (1978), 11, 343; Liu et. al., J. Org. Chem. (1996), 61, 6693-99; van Kerckhoven et. al., Macromolecules (1972), 5, 541; Schilling et. al. Macromolecules (1969), 2, 85; and Puetter et. al., J. Prakt. Chem. (1951), 149, 183. Furans can be prepared using conventional methods such as those shown in the following references and references cited therein: Feldman et. al., Tetrahedron Lett. (1992), 47, 7101, McDonald et. al., J. Chem. Soc. Perkin Trans. (1979), 1 1893. Pyrazines can be prepared using methods such as those shown in Turchi et. al., Tetrahedron (1998), 1809, and references cited therein.

In a preferred embodiment of the invention employing mixtures of the present monomers and other monomers as previously disclosed, it is desirable to maintain a ratio of the corresponding A-functionality and B-functionality in the mixture such that the ratio of B-functional groups to A-functional groups in the reaction mixture is in the range of 1:10 to 10:1, and most preferably from 2:1 to 1:4. Preferably, the composition additionally comprises a solvent and optionally may also comprise a poragen.

Suitable poragens that may be separately added to a composition herein or bonded to the monomer include any compound that can form small domains in a matrix formed from the monomers and which can be subsequently removed, for example by thermal decomposition. Preferred poragens are polymers including homopolymers and interpolymers of two or more monomers including graft copolymers, emulsion polymers, and block copolymers. Suitable thermoplastic materials include polystyrenes, polyacrylates, polymethacrylates, polybutadienes, polyisoprenes, polyphenylene oxides, polypropylene oxides, polyethylene oxides, poly(dimethylsiloxanes), polytetrahydrofurans, polyethylenes, polycyclohexylethylenes, polyethyloxazolines, polyvinylpyridines, polycaprolactones, polylactic acids, copolymers of the monomers used to make these materials, and mixtures of these materials. The thermoplastic materials may be linear, branched, hyperbranched, dendritic, or star-like in nature. The poragen may also be designed to react with the cross-linkable matrix precursor or oligomer during or subsequent to b-staging to form blocks or pendant substitution of the polymer chain. For example, thermoplastic polymers containing reactive groups such as vinyl, acrylate, methacrylate, allyl, vinyl ether, maleimido, styryl, acetylene, nitrile, furan, cyclopentadienone, perfluoroethylene, BCB, pyrone, propiolate, or ortho-diacetylene groups can form chemical bonds with precursor compounds containing suitable reactive groups, such as bromo-, vinyl- or ethynyl functionality.

Suitable block copolymer poragens include those wherein one of the blocks is compatible with cross-linked polymer matrix resin and the other block is incompatible therewith. Useful polymer blocks can include polystyrenes such as polystyrene and poly-α-methylstyrene, polyacrylonitriles, polyethylene oxides, polypropylene oxides, polyethylenes, polylactic acids, polysiloxanes, polycaprolactones, polyurethanes, polymethacrylates, polyacrylates, polybutadienes, polyisoprenes, polyvinyl chlorides, and polyacetals, and amine-capped alkylene oxides (commercially available as Jeffamine™ polyether amines from Huntsman Corp.).

Highly preferred poragens are crosslinked polymers made by solution or emulsion polymerization. Such polymerization techniques are known in the art, for example, EP-A-1,245,586, and elsewhere. Very small crosslinked hydrocarbon based polymer particles have been prepared in an emulsion polymerization by use of one or more anionic-, cationic-, or non-ionic surfactants. Examples of such preparations may be found in J. Dispersion Sci. and Tech., vol. 22, No. 2-3, 231-244 (2001); “The Applications of Synthetic Resin Emulsions”, H. Warson, Ernest Benn Ltd., 1972, p. 88; Colloid Polym. Sci., 269, 1171-1183 (1991), Polymer. Bull., 43, 417-424 (1999), PCT 03/04668, filed Feb. 12, 2003 and U.S. Ser. No. 10/366,494, filed Feb. 12, 2003, among other sources.

Preferably, the monomer is chemically bound or grafted to the porogen by a palladium catalyzed reaction of an ethynyl terminated poragen precursor with an aromatic halogen containing diketone or diaryl-substituted acetone derivative. This may be best accomplished by incorporating the functionalized porogen in the monomer prior to b-staging. In this maner, the bound poragen is uniformly incorporated into the resulting cured polymer. The mixture is then coated onto a substrate (preferably solvent coated as for example by spin coating or other known methods). The matrix is cured and the bound porogen is removed, preferably by heating to a temperature above the thermal decomposition temperature of the poragen. This results in uniform, extremely small poragens in the resin, and uniform, extremely small pores (nanopores) in the vitrified resin matrix. Porous films prepared in this manner are useful in making integrated circuit articles where the film separates and electrically insulates conductive metal lines from each other.

Porous Matrix from AxByP*z Monomers and Oligomers

The poragen is desirably a material that, upon removal, results in formation of voids or pores in the matrix having an average pore diameter from 1 to 200 nm, more preferably from 2 to 100 nm, most preferably from 5 to 50 nm. Desirably, the pores are not interconnected, that is the resulting matrix has a closed cell structure. The nature of the bound poragen is chosen based on a number of factors, including the size and shape of the pore to be generated, the method of poragen decomposition, the level of any poragen residue permitted in the porous nanostructure, and the reactivity or toxicity of any decomposition products formed. It is also important that the matrix have enough crosslinking density to support the resulting porous structure.

In particular, the temperature at which pore formation occurs should be carefully chosen to be sufficiently high to permit prior solvent removal and at least partial vitrification of the b-staged oligomer, but below the glass temperature, Tg, of the vitrified matrix. If pore formation takes place at a temperature at or above the Tg of the matrix, partial or full collapse of the pore structure may result.

Examples of suitable bound poragens for use herein include moieties having different macromolecular architectures (linear, branched, or dendritic) and different chemical identities, including polyacrylates, polymethacrylates, polybutadiene, polyisoprenes, polypropylene oxide, polyethylene oxide, polyesters, polystyrene, alkyl-substituted polystyrene, and all copolymer combinations, including block copolymers, and functionalized derivatives thereof. Preferably, substances used to prepare bound poragens have one or more functional groups by means of which the poragen is chemically bonded to the monomer during preparation. Suitable functionalized polymeric substances include, ethynyl capped polystyrene, ethynyl capped crosslinked polystyrene copolymers, ethynyl capped polystyrene bottlebrush, and ethynyl capped polystyrene star shaped polymers. Most preferably, the bound poragen forming compound is a crosslinked vinyl aromatic microemulsion particle (MEP) containing addition polymerizable ethynyl functional groups.

MEPs are intramolecularly crosslinked molecular species of extremely small particle size possessing a definable surface of approximately spherical shape. Highly desirably, the MEP's have an average particle size from 5 to 100 nm, most preferably from 5 to 20 nm Desirably the grafting level of the functionalized MEP is sufficient to result in self-alignment, thereby resulting in discrete microphase separation of the MEP's. Upon thermal treatment, the MEP phase may decompose while cross-linking of A and B functionality of the monomer proceeds, thereby forming cross-linked oligomers or vitrified solids with homogeneously distributed, extremely small (<10 nanometers average size) voids in a single step.

The result of incorporating bound poragens into the matrix during its formation in the foregoing manners is a near uniform correspondence of pores with initial bound poragen moieties and limited or no agglomeration and heterogeneous phase separation of the poragens. In addition, separate thermal processing for purposes of pore formation may be avoided if the decomposition temperature of the bound poragen is appropriately chosen. The resultant articles, including films or coatings, are extremely low dielectric constant, nanoporous materials having highly uniform electrical properties due to the uniformity of pore distribution.

Highly desirably, the matrix materials formed from monomers of the present invention are relatively thermally stable at temperatures of at least 300° C., preferably at least 350° C. and most preferably at least 400° C. In addition, the matrix polymer also has a Tg of greater than 300° C. and more preferably greater than 350° C. after being fully crosslinked or cured. Further desirably, the crosslinking or vitrification temperature of the invention, defined as the temperature upon heating at which flexural modulus increases most quickly, is desirably below the decomposition temperature of the poragen, preferably less than or equal to 400° C., most preferably, less than or equal to 300° C. This property allows crosslinking to take place before substantial pore formation occurs, thereby preventing collapse of the resulting porous structure. Finally, in a desirably embodiment of the invention, the flexural modulus of the partially crosslinked and cured polymer, either with or without poragen present, desirably reaches a maximum at temperatures less than or equal to 400° C., preferably less than or equal to 350° C., and most preferably, less than or equal to 300° C. and little or no flexural modulus loss occurs upon heating the fully cured matrix to a temperature above 300° C., such as may be encountered during pore formation via thermolysis.

In one suitable method of operation, monomer, optional poragen forming material, and optional solvent are combined and heated at elevated temperature, preferably at least 160° C., more preferably at least 200° C. for at least several hours, more preferably at least 24 hours to make a solution of crosslinkable b-staged oligomers bearing bound poragens. The amount of monomer relative to the amount of separately added poragen may be adjusted to give a cured matrix having the desired porosity. Alternatively, a comonomer with or without bound poragen may be included in the polymerizable composition to control the quantity of pores in the resulting matrix. Preferably, the amount of bound poragen based on total monomer weight is from 5 to 80 percent, more preferably from 20 to 70 percent, and most preferably from 30 to 60 percent.

Solutions containing bound poragen monomer for use herein desirably are sufficiently dilute to result in optical clear solutions having the desired coating and application properties. Preferably, the amount of solvent employed is in the range of 50-95 percent based on total solution weight. The solution may be applied to a substrate by any suitable method such as spin coating, and then heated to remove most of the remaining solvent and leave the monomer or b-staged oligomer, containing bound poragen moieties dispersed therein. During the solvent removal process and/or during subsequent thermal processing, the poragen phase desirably forms separate uniformly dispersed occlusions in a fully cured or cross-linked matrix. Upon continued or subsequent heating, the occlusions decompose into decomposition products that may diffuse through the cured matrix, thereby forming a porous matrix.

The concentration of pores in above porous matrix is sufficiently high to lower the dielectric constant or reflective index of the cured polymer, but sufficiently low to allow the resulting porous matrix to withstand the process steps required in the fabrication of microelectronic devices. Preferably, the quantity of pores in the resulting cross-linked porous matrix is sufficient to result in materials having a dielectric constant of less than 2.5, more preferably less than 2.0.

The average diameter of the pore is preferably less than 100 nm, more preferably less than 20 nm, and most preferably less than 10 nm. The pore sizes can be easily controlled by adjusting the size of the MEP employed in preparing the monomers of the invention.

The compositions of the invention may be used to make dielectric films and interlayer dielectrics for integrated circuits in accordance with known processes, such as those of U.S. Pat. No. 5,965,679. To make a porous film the bound poragen is preferably removed by thermal decomposition.

The invention is further illustrated by the following Examples that should not be regarded as limiting of the present invention. Unless stated to the contrary or conventional in the art, all parts and percents are based on weight.

### EXAMPLE 1

### Synthesis of A₂BP*₂ Monomer

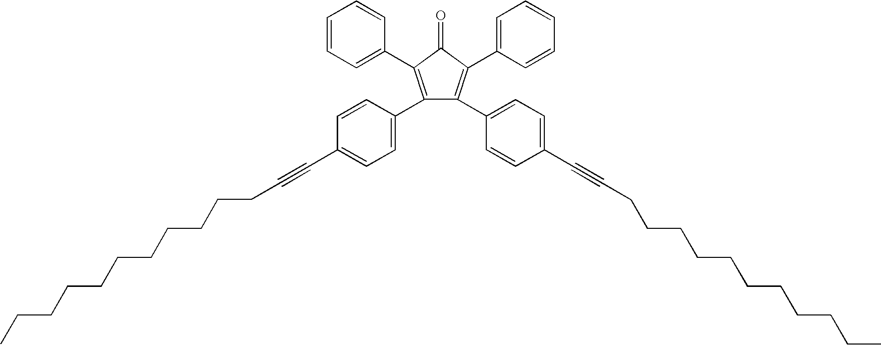

A) Synthesis of 4,4′-decyl-ethynyllbenzil

To a 100 ml round flask are added 4,4′-dibromobenzil (7.36 g, 0.02 mole), DMF (50 ml), dodecyne (8.3 g, 0.05 mole), and triethylamine (10.1 g, 0.1 mole). The resulting mixture is purged with nitrogen for 15 minutes, and then triphenylphosphine (0.47 g) and palladium acetate (0.0067 g) are added. The reaction mixture is heated to 70° C. for 7 hours. After cooling to room temperature, water (100 ml) is added. The crude product is filtered and the solid redissolved into methylene chloride. Upon evaporation of the solvent, yellow crystals are obtained which are further recrystallized from methylene chloride/methanol. Yield 9.3 g, 86 percent.

### B) Monomer Synthesis

4,4′-decylethynylbenzil (2.69 grams, 5.0 mmole) and 1.26 grams (6.0 mmole) of 3,3′-diphenyl-2-propanone are added to a reactor containing 100 mL of anhydrous 2-propanol. Stirring and heating are commenced, and once the suspension reaches reflux temperature, tetrabutylammonium hydroxide (50 percent in water, 0.25 mL in two portions) is added, immediately inducing a deep red purple color. After maintaining at reflux for 1.5 hours, HPLC analysis indicates that full conversion of 4,4′-decylethynylbenzil reactant is achieved. At this time, the oil bath is removed from the reactor, and the reaction mixture is allowed to cool to 40° C. The product is recovered via filtration through a medium fritted glass funnel. The crystalline product on the funnel is washed with two 20 mL portions of 2-propanol, then dried in a vacuum oven to provide 2.0 grams of the desired A₂BP*₂ monomer. DSC analysis shows a melting point of 71.5° C. with an onset temperature for the Diels-Alder reaction of 196° C. The Diels-Alder reaction reaches a maximum in the DSC curve at 248° C. and ends at 315° C. with a total heat output of 154 J/g.

### EXAMPLE 2

### Preparation of Porous Matrix Formulation

To a 50 ml round flask was added 2.0 g of bound poragen containing monomer from Example 1 and 5.0 g of γ-butyrolactone (GBL). The resulting mixture is purged under nitrogen for 15 minutes and then heated to 200° C. with an oil bath under nitrogen for 6 hours. The mixture is then cooled to 145° C. and diluted with 3.3 g of cyclohexanone. The mixture is cooled to room temperature to give a solution of b-staged polymer.

What is claimed is:

1. A compound comprising i) one or more dienophile groups (A-functional groups), ii) one or more ring structures comprising two conjugated carbon-to-carbon double bonds and a leaving group L (B-functional groups), and iii) one or more chemically bound, polymeric or oligomeric poragens, P*, characterized in that the A-functional group of one monomer is capable of reaction under cycloaddition reaction conditions with the B-functional group of a second monomer to thereby form a cross-linked, polyphenylene polymer comprising chemically bound porogens.

2. A compound according to claim 1 corresponding to the formula, 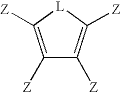
wherein L is —O—, —S—, —N═N—, —(CO)—, —(SO₂)—, or —O(CO)—;
Z is independently in each occurrence hydrogen, halogen, an unsubstituted or inertly substituted hydrocarbyl group Z″, or two adjacent Z groups together with the carbons to which they are attached form a fused aromatic ring,
Z″ is a divalent derivative of an unsubstituted or inertly substituted hydrocarbyl group joining two or more of the foregoing structures, or joining an A-functionality, a bound poragen or a combination of the foregoing,
and in at least one occurrence, Z is -Z″-C—CP*; or
in at least one occurrence, Z is -Z″-C≡CR and in at least one other occurrence Z is a bound poragen; wherein,
P* is independently each occurrence a bound poragen; and
R is independently each occurrence selected from the group consisting of hydrogen, C₁₋₄ alkyl, C₆₋₆₀ aryl, and C₇₋₆₀ inertly substituted aryl groups.

3. A compound according to claim 1 corresponding to the formula: 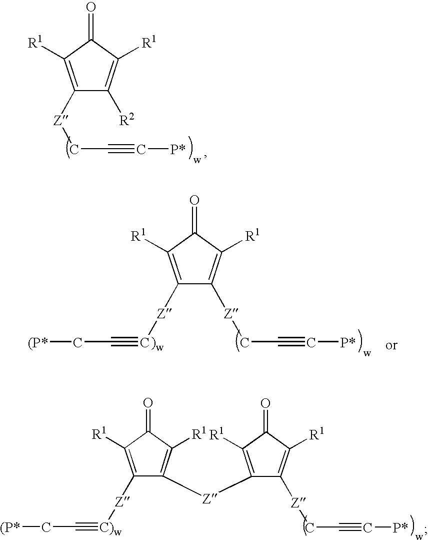
wherein R¹ is P*, C₆₋₂₀ aryl or inertly substituted aryl, or R²OC(O)—;
R² is P*, C₆₋₂₀ aryl or inertly substituted aryl;
w independently each occurrence is an integer from 1 to 3
Z″ is a divalent aromatic group, and
P* is a bound poragen comprising a monovalent derivative of a linear or branched oligomer or polymer of a vinylaromatic monomer, alkylene oxide, arylene oxide, alkylacrylate or alkylmethacrylate, or a cross-linked derivative thereof.

4. A compound according to claim 1 corresponding to the formula: 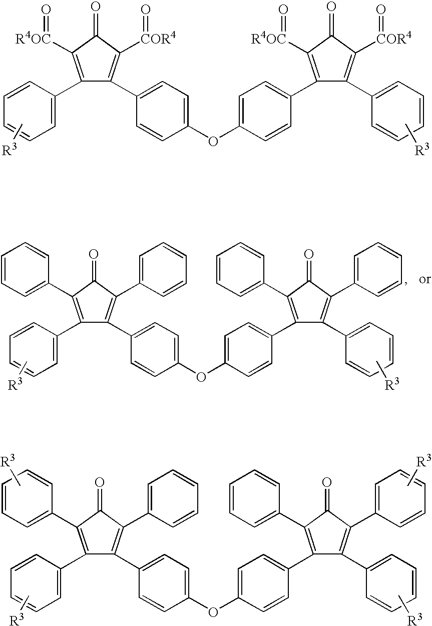
wherein R³ each occurrence is —C≡C—P*, and
R⁴ independently each ocurrence is H, phenyl or P*.

5. A cross-linked polymer formed by curing a composition comprising a compound according to any one of claims 1-4.

6. A cross-linked polymer according to claim 5 comprising a bound poragen, P*.

7. A porous matrix formed by removing the bound poragen from the cross-linked polymer of claim 6.
